(19) United States
(12) Patent Application Publication
(10) Pub. No.: US 2006/0184027 A1
(43) Pub. Date: Aug. 17, 2006

(54) Diagnostic imaging system, magnetic resonance imaging apparatus, and method of diagnostic imaging

(75) Inventors: Yoshiteru Watanabe, Nasushiobara-Shi (JP); Yukihiko Tomoda, Otawara-Shi (JP); Hideyuki Ooba, Nasushiobara-Shi (JP)

Correspondence Address: NIXON & VANDERHYE, PC, 901 NORTH GLEBE ROAD, 11TH FLOOR, ARLINGTON, VA, 22203, US

(73) Assignees: Kabushiki Kaisha Toshiba, Tokyo (JP); Toshiba Medical Systems Corporation, Otawara-shi (JP)

(21) Appl. No.: 11/322,212

(22) Filed: Jan. 3, 2006

(30) Foreign Application Priority Data
Jan. 6, 2005 (JP) P2005-1785

(51) Int. Cl. A61B 8/14

(52) U.S. Cl. 600/440

(57) ABSTRACT

A diagnostic imaging system includes a scanner, a plurality of computers, and a control unit. The scanner scans a subject to obtain data. The control unit switches between a multiprocessing mode and a parallel processing mode, the multiprocessing mode allowing the same process in any of a medical-image forming process based on the data obtained and the subsequent processes to be executed by two or more computers of the plurality of computers, and the parallel processing mode allowing a plurality of different processes in the medical-image forming process and the subsequent processes to be executed concurrently by two or more computers of the plurality of computers.

### BACKGROUND OF THE INVENTION

1. Field of the Invention

The present invention relates to a diagnostic imaging system, a magnetic resonance imaging apparatus, and a method of diagnostic imaging that collects data obtained by scanning a subject to form a medical image on the basis of the collected data.

2. Description of the Related Art

Diagnostic imaging systems that include a diagnostic imaging apparatus, such as a magnetic-resonance imaging (MRI) apparatus, an X-ray CT scanner, a single-photon-emission computed tomography (SPECT) apparatus, or a positron-emission tomography (PET) apparatus, are recently used widely in medical institutions. JP-A-2004-208954 and JP-A-2004-305293 disclose the structure of conventional diagnostic imaging systems.

The diagnostic imaging system disclosed in JP-A-2004-208954 includes a scanner (gantry) that scans a subject and an operating console for remotely controlling the scanner. The operating console stores a computer program that gives an instruction to the scanner, a computer program for reconstructing a medical image on the basis of data obtained by the scanner, and other programs. The diagnostic imaging system disclosed in 2004-305293 includes a scanner, an operating console, and an image reconstructing unit.

Such conventional diagnostic imaging systems have the problems that, if the operating console or the image reconstructing unit fails in software or hardware, the process of data collection or image reconstruction stops urgently to hinder smooth examination and to impose a burden of reexamination on the subject. Particularly, certainty is required for processes that permit no reexamination such as a data-collection process using a contrast medium, a collection and reconstruction process in displaying an image in real time during examination, a long-time collection process, and a collection and reconstruction process for emergency patients having a restriction in time. If such processes are interrupted during execution, a severe burden is imposed on the patients, and in some cases, a serious life-threatening situation will happen.

When the operating console and so on fail to stop the process urgently, records (logs) on the details of the process and the processing state of various set data are not often kept and as such, it sometimes become difficult to track down and solve the essential cause of the anomaly. Such situations will lead to inconvenience in offering effective solutions to the anomaly when using the remote-maintenance service disclosed in, e.g., JP-A-2003-190105.

Furthermore, for application of the structure disclosed in JP-A-2004-208954, in which a single computer performs all the processes, if the computer becomes infected with computer virus, all the processes become unavailable. For application of the structure disclosed in JP-A-2004-305293, in which a plurality of computers shares the processes, all the computers have a significant danger of stopping at a time, because the computers generally have the same operating system (OS). The plurality of computers often has the same hardware structure, so that all the computers can fail if one component fails.

In addition to the processes for which certainty is required, the processes of the diagnostic imaging system include processes for which high speed is required (a normal reconstruction process, general image processings, an image storing process, a transfer process, and a film-layout producing process). However, the conventional systems could not switch the mode of execution to the process-by-process requests.

### SUMMARY OF THE INVENTION

The invention has been made in view of such circumstances. Accordingly, it is an object of the invention to provide a diagnostic imaging system, a magnetic resonance imaging apparatus, and a method of diagnostic imaging in which processes for which certainty is required can be executed with certainty even if an anomaly occurs, and processes for which high speed processing is required can be executed rapidly.

Another object of the invention is to provide a system and method of diagnostic imaging capable of recording the processing state when the system fails and performing effective and rapid maintenance.

In order to achieve the above objects, a diagnostic imaging system according to a first aspect of the invention includes: a scanner that scans a subject to obtain data; a plurality of computers; and a control unit that switches between a multiprocessing mode and a parallel processing mode, the multiprocessing mode allowing the same process in any of a medical-image forming process based on the data obtained and the subsequent processes to be executed by two or more computers of the plurality of computers, and the parallel processing mode allowing a plurality of different processes in the medical-image forming process and the subsequent processes to be executed concurrently by two or more computers of the plurality of computers.

In order to achieve the above objects, it is preferable that the diagnostic imaging system further include: an anomaly-detection unit that detects whether an anomaly has occurred in the processing state of the process in any of the medical-image forming process and the subsequent processes by the two or more computers; and a processing-state recording unit that records the processing state when the anomaly is detected together with anomaly-detection information, wherein when a new process for the processing state recorded together with the anomaly-detection information is executed, the control unit allows the two or more computers to execute the new process in the multiprocessing mode.

In order to achieve the above objects, a magnetic resonance imaging apparatus according to a second aspect of the invention includes: a scanner that scans a subject to obtain data; a plurality of computers; an exposure-condition acquisition unit that acquires exposure conditions of the scan; and a control unit that switches between a multiprocessing mode and a parallel processing mode depending on the exposure conditions, the multiprocessing mode allowing the same process in any of a medical-image forming process based on the data obtained and the subsequent processes to be executed by two or more computers of the plurality of computers, and the parallel processing mode allowing a plurality of different processes of any of the medical-image forming process and the subsequent processes to be executed concurrently by two or more computers of the plurality of computers.

In order to achieve the above objects, a method of diagnostic imaging according to a third aspect of the invention includes the steps of: obtaining data by scanning a subject; and switching between a multiprocessing mode and a parallel processing mode, the multiprocessing mode allowing the same process in any of a medical-image forming process based on the data obtained and the subsequent processes to be executed by two or more computers of the plurality of computers, and the parallel processing mode allowing a plurality of different processes in the medical-image forming process and the subsequent processes to be executed concurrently by two or more computers of the plurality of computers.

In order to achieve the above objects, it is preferable that the method of diagnostic imaging further include the steps of: detecting whether an anomaly has occurred in the processing state of the process in any of the medical-image forming process and the subsequent processes by the two or more computers; and recording the processing state when the anomaly is detected together with anomaly-detection information, wherein when a new process for the processing state recorded together with the anomaly-detection information is executed, the two or more computers are allowed to execute the new process in the multiprocessing mode.

The diagnostic imaging system, the magnetic resonance imaging apparatus, and the method of diagnostic imaging according to the invention allows sure processing of a process for which certainty is required even if anomaly occurs, and rapid processing of a process for which high-speed processing is required.

The system and method of diagnostic imaging according to the invention allows recording of a processing state when the system fails, allowing effective and rapid maintenance.

### BRIEF DESCRIPTION OF THE DRAWINGS

FIG. 1 is a schematic diagram of an example of the overall structure of a diagnostic imaging system according to an embodiment of the present invention;

FIG. 2 is a schematic block diagram of an example of the outline of a plurality of computers incorporated in the diagnostic imaging system according to the embodiment of the invention;

FIG. 3 is a schematic block diagram of an example of the outline of a management computer incorporated in the diagnostic imaging system according to the embodiment of the invention;

FIG. 4 is a flowchart of an example of the procedure executed by the diagnostic imaging system according to the embodiment of the invention;

FIG. 5 is a flowchart of an example of the procedure executed by the diagnostic imaging system according to the embodiment of the invention; and

FIG. 6 is a flowchart of an example of the procedure executed by a modification of the diagnostic imaging system of the invention.

### DESCRIPTION OF THE PREFERRED EMBODIMENT

A diagnostic imaging system, a magnetic resonance imaging apparatus, and a method of diagnostic imaging according to a preferred embodiment of the invention will be specifically described with reference to the drawings.

1. System Structure

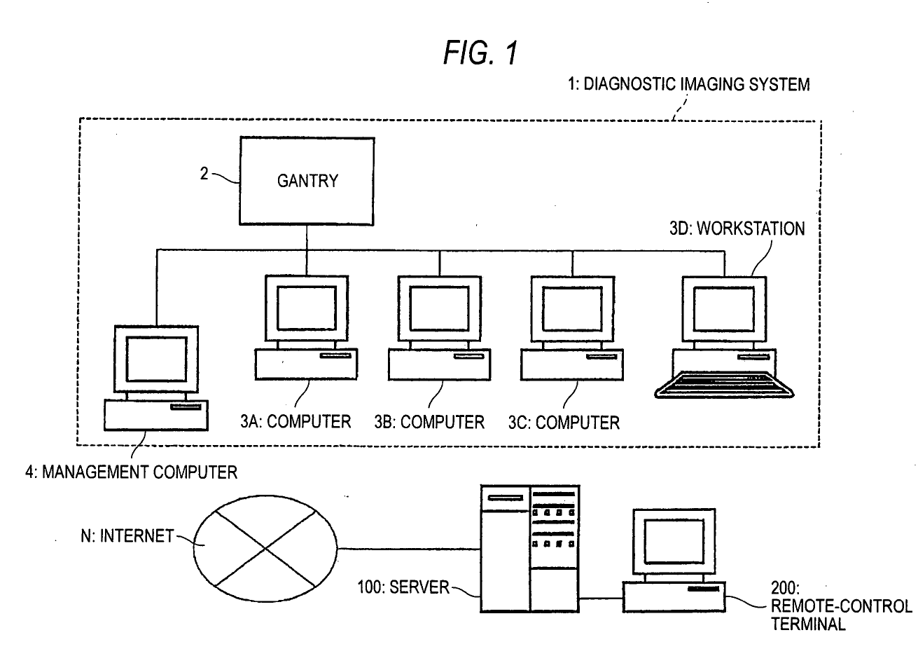

FIG. 1 is a schematic diagram of an example of the overall structure of a diagnostic imaging system according to an embodiment of the present invention. In FIG. 1 the diagnostic imaging system 1 is operated in various medical institutions including hospitals and laboratories, and includes a gantry 2 that scans a subject (not shown); computers 3A to 3C and a workstation 3D that execute a medial-image forming process based on data obtained by the scanning of the gantry 2 and a post process for the medical images formed; and a management computer 4 that controls the operation of the computers 3A to 3C and the workstation 3D. The gantry 2, the computers 3A to 3C, the workstation 3D, and the management computer 4 are connected to one another via a local network such as a LAN or a dedicated line.

The management computer 4 has a communication device (a communicating section 43, to be described later) such as a modem for communicating with a server 100 via the Internet N. The server 100 is installed in a maintenance service company together with a remote-control terminal 200, and manages various data on the remote maintenance of the diagnostic imaging system 1. The remote-control terminal 200 is a computer for use by service engineers of the maintenance service company. The remote-control terminal 200 is mainly used for monitoring the operation of the diagnostic imaging system 1. The server 100 and the remote-control terminal 200 are examples of “a remote computer”.

Gantry

The gantry 2 is a scanner that scans a subject to obtain data indicative of the form and function of the body. The gantry 2 can be any diagnostic imaging apparatus: e.g., a magnetic-resonance imaging (MRI) apparatus, an X-ray computed tomography (CT) scanner, a single-photon-emission computed tomography (SPECT) apparatus, a positron-emission tomography (PET) apparatus, an ultrasonic diagnostic imaging apparatus, and a computed radiography (CR) apparatus.

Computer and Workstation

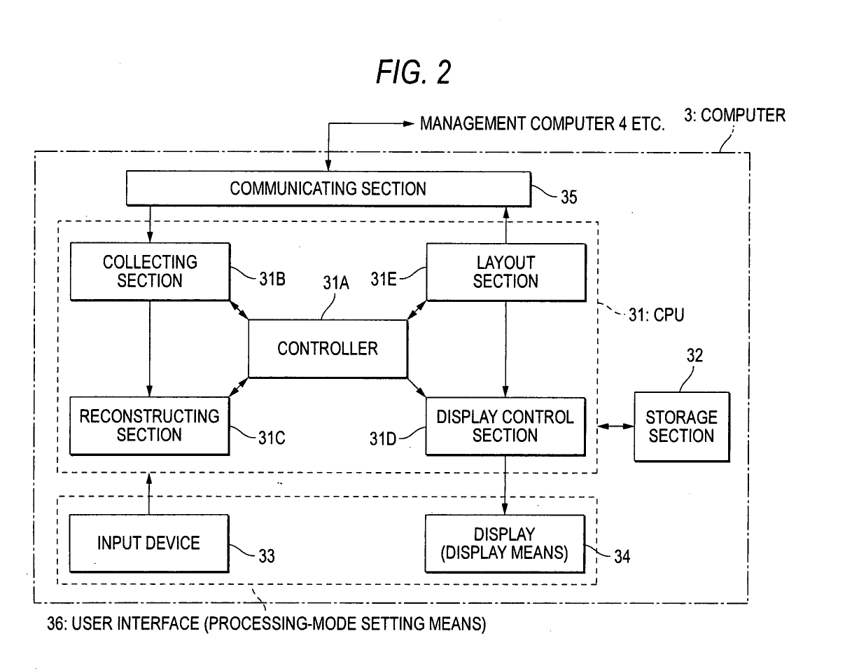

The structure of the computers 3A to 3C and the workstation 3D (collectively referred to as computers 3) will now be described. The computers 3A to 3C are each constructed of a commercial personal computer, for example. The workstation 3D is constructed of a general-purpose workstation, for example. FIG. 2 is a schematic block diagram of an example of the outline of the control system of the computers 3.

The computers 3 each include a CPU 31 serving as an operating and controlling processor; a storage section 32 including storage devices such as a ROM, a hard disk drive, and an image memory; an input device 33 such as a keyboard, a mouse, a trackball, and a control panel; a display 34 that displays various screens, such as an LCD or a CRT; and a communicating section 35 that transfers data via a local network such as a LAN.

The input device 33 and the display 34 constitute the user interface 36 of the computers 3. The user interface 36 corresponds to an example of “a processing-mode setting unit” for setting processing modes (see later). The user interface 36 may not necessarily be equipped in all the computers 3 but may be provided in any of them (e.g., the workstation 3D).

The communicating section 35 includes a network adaptor for connecting to a LAN or the like. The communicating section 35 transfers data with the management computer 4, the other computers 3, a film printer (imager) and an archive database (both are not shown).

The CPU 31 includes a controller 31A for controlling the components of the computer 3 and for arithmetic processing. The controller 31A executes the control and arithmetic processing according to a computer program (not shown) stored in the storage section 32. The CPU 31 further includes a collecting section 31B that collects data obtained from the gantry 2; a reconstructing section 31C that reconstructs a medical image on the basis of the collected data; a display control section 31D that allows a display 34 to display various images such as medical images; and a layout section 31E that sets a layout when printing the medical image on a film.

An example of the film-layout setting process and the film printer is disclosed, for example, in JP-A-2001-292295 by the applicant. The computers 3 have a preprocessing function (not shown) of performing various corrections for the data obtained by the gantry 2.

At least one (particularly, the workstation 3D) of the computers 3 is used as the console of the gantry 2. The computer 3 stores a control program for controlling the operation of the gantry 2, thus controlling the gantry 2 according to input from the input device 33. At that time, the display 34 displays a predetermined operation screen.

It is preferable that the computers 3A to 3C and the workstation 3D have different operating systems (OSs). For example, the computers 3A to 3C are equipped with Windows™; the workstation 3D is equipped with UNIX™. While the “plurality of computers” can have any desired hardware structure, it is preferable that they have different hardware structures. The embodiment has general personal computers 3A to 3C and a workstation 3D different from those. The number of computers that execute the process based on the data obtained by the gantry 2 can be set freely.

Management Computer

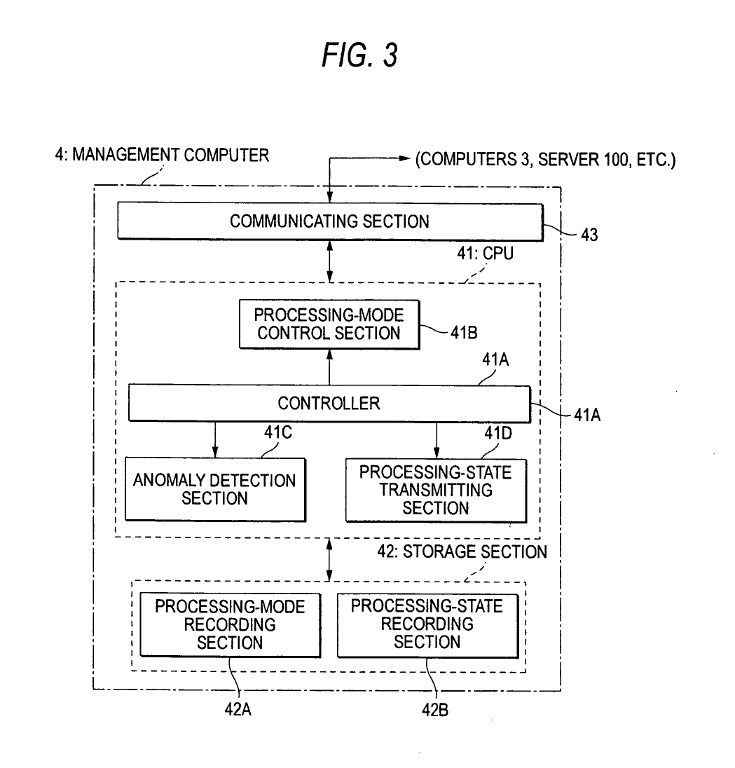

The structure of the management computer 4 will be described. FIG. 3 is a schematic block diagram of an example of the control system of the management computer 4.

The management computer 4 includes a CPU 41 serving as an operating and controlling processor; a storage section 42 including storage devices such as a ROM, a hard disk drive, and an image memory; and a communicating section 43 that transfers data via a local network such as a LAN or a wide-area network such as the Internet N.

The CPU 41 includes a controller 41A for controlling the components of the management computer 4 and for arithmetic processing. The controller 41A executes the control and arithmetic processing according to a computer program (not shown) stored in the storage section 42. The CPU 41 further includes a processing-mode control section 41B; an anomaly detection section 41C; and a processing-state transmitting section 41D.

The processing-mode control section 41B is an example of “a control unit”, which switches the processing modes of the computers 3A to 3C and the workstation 3D by controlling the operations thereof. The processing modes indicate the states of the execution of the processes by the computers 3. The processing modes achieved by the processing-mode control section 41B include a multiprocessing mode in which two or more computers 3 execute the same process and a parallel processing mode in which two or more computers 3 execute different processes.

The multiprocessing mode allows the same process to be executed by two or more computers 3 and as such, even if one of the computers 3 fails, the execution results of the other computers 3 can be used. Accordingly, this mode is preferable for processes for which certainty is required, such as when redoing is not allowed. Such processes include a data-collection process using a contrast medium, a collection and reconstruction process when displaying an image in real time during examination, a long-time collection process, and a collection and reconstruction process for emergency patients having a restriction in time.

The parallel processing modes include, e.g., a processing mode in which one process is divided into two or more partial processes, and one partial process is executed by one computer 3, and a processing mode in which a series of multiple processes are shared by two or more computers 3. Such parallel processing modes are suitable for high-speed processing. The processes for which high-speed processing is required include, e.g., a normal reconstruction process, general image processing, an image storage process (archiving), an image transfer process, and a film-layout process.

The processing-mode control section 41B can be constructed to switch between the multiprocessing mode and the parallel processing mode depending on the conditions of exposure as necessary. In this case, the processing-mode control section 41B provides information to the display 34 of the computer 3 via the network to display an exposure-condition input screen. Thus, the user can notify the processing-mode control section 41B of the exposure conditions by operating the input device 33. That is to say, the processing-mode control section 41B can be provided with the function of obtaining exposure conditions.

Alternatively, the processing-mode control section 41B may obtain exposure conditions from the other component such as the gantry 2 via a network, or through the user interface of the management computer 4.

The processing-mode control section 41B previously stores an exposure-condition table showing the relationship among various exposure conditions and information indicative of whether the processing mode is the multiprocessing mode or the parallel processing mode, or whether the processing mode is the option of the user. The processing-mode control section 41B can therefore set a processing mode according to exposure conditions obtained with reference to the exposure-condition table.

As the exposure conditions to be referred to for switching the processing mode, it is practical to use exposure conditions that allow determination whether the exposure requires certainty. Particularly when the gantry 2 is an MRI apparatus, a decrease in throughput due to reexposure can be prevented in such a way that the processing mode is switched to the multiprocessing mode with reference to exposure conditions indicative of whether the exposure uses a contrast medium and whether the exposure is long-time exposure, such as a dynamic scan, a whole-body scan, a moving-bed scan that is a scan with a moving bed, angio imaging, and diffusion imaging.

A more specific exposure condition is a pulse sequence that is one of the MRI exposure conditions. For example, when the diffusion sequence or the pulse sequence for angio imaging is used for exposure, the processing-mode control section 41B is given the information indicative of that the sequence has been set through the input device 33 of the computer 3 or from the gantry 2 via a network so that the processing-mode control section 41B can select the multiprocessing mode.

In addition, the processing-mode control section 41B can execute the processes by two or more computers 3 at the same timing by outputting a synchronizing signal to synchronize the processes of the computers 3.

The processing-mode control section 41B operates according to the set processing mode to execute the following operations, to be described below as appropriate, in addition to the foregoing operations.

The anomaly detection section 41C will then be described. The anomaly detection section 41C detects whether the computers 3A to 3C and the workstation 3D fail. The anomaly detection section 41C particularly detects whether the computer 3 in operation fails.

An example of the anomaly detection process will be described. The anomaly detection section 41C first sends an anomaly check signal to the computers 3 in operation. The anomaly check signal is sent, for example, at regular intervals. Upon reception of the anomaly check signal, the computers 3 returns data indicative of the state of the process executed at the reception (referred to as processing-state data) to the management computer 4.

The processing-state data contains the details of the process during execution (e.g., a collecting process and a reconstructing process) and set data for the process (e.g., the kind of a reconstruction function used in the reconstructing process), and the progress of the process at the time processing-state data is generated (e.g., which image in slice position is being reconstructed during the reconstructing process). The processing-state data is generated by the CPU on the basis of the log of the execution state of the computer program by the CPU of the computer 3 that is executing the process.

The anomaly detection section 41C detects the presence or absence of the anomaly (particularly, synchronous anomaly) of the state of process by the computers 3 on the basis of the state of returning of processing-state data in response to the anomaly check signal. For example, a computer 3 to which no processing-state data is returned in a specified time from the transmission of the anomaly check signal, and a computer 3 to which a code indicative of noncommunication or the like is returned may have synchronous anomaly or freeze due to a decrease in processing speed. When there are computers 3 to which different processing-state data (data on the progress of the process) is returned during the multiprocessing mode, this indicates that two or more computers 3 have a synchronous anomaly. Also when hardware failure and computer-virus infection cause the foregoing returning situation and as such, the anomaly detection section 41C detects an anomaly in the processing state of the computer 3.

The processing-mode control section 41B records the processing-state data returned from the computers 3 in a processing-state recording section 42B of a storage section 42 for each computer 3. The recorded processing-state data is stored as the process record (log) of each computer 3. The log contains the time the processing-state data is generated and the time anomaly is detected or not detected as in general. When the computer 3 operates normally, the log may be recorded in the storage section 32 one by one.

When an anomaly is detected by the anomaly detection section 41C, the processing-mode control section 41B attaches error information (anomaly-detection information), such as a flag indicative of the detection of the anomaly, to the processing-state data of the computer 3 in which the anomaly is detected, and records it in the processing-state recording section 42B.

In the anomaly checking process, processing-state data is returned in response to the anomaly check signal from the anomaly detection section 41C. Alternatively, the computers 3 in operation may transmit processing-state data regularly to the management computer 4. It is also possible that the computers 3 store processing-state data regularly in the storage section 32.

The processing-state transmitting section 41D controls the communicating section 43 to transmit data on the processing state of the computers 3 to a server 100 via the Internet N. Particularly, the processing-state transmitting section 41D transmits processing-state data when an anomaly is detected by the anomaly detection section 41C to the server 100 together with error information.

The processing-state data can be sent to the server 100 at any timing; for example, it may be sent directly after an anomaly is detected, or alternatively, it may be sent at a preset transmission time. In the former, when an anomaly is detected, the processing-mode control section 41B attaches error information to the processing-state data at the detection of the anomaly, and the processing-state transmitting section 41D transmits the processing-state data and the error information to the server 100. On the other hand, when processing-state data is temporarily recorded in the processing-state recording section 42B as in the latter, the processing-state transmitting section 41D acquires the processing-state data to which the error information is attached from the processing-state recording section 42B at a specified transmission time, and sends it to the server 100. The transmission timing is not limited to those; for example, the processing-state data can be sent in response to the request of the operator of a remote-control terminal 200 or the computer 3.

The storage section 42 includes a processing-mode recording section 42A and a processing-state recording section 42B. The recording sections 42A and 42B are constituted of a storage region constructed in the storage section 42 in advance.

The processing-mode recording section 42A records processing modes set for the various processes executed by the computers 3: for example, a default processing mode; a processing mode manually set through the user interface 36; and a processing mode automatically set by the processing-mode control section 41B.

The processing-state recording section 42B records the processing-state data and error information of the computers 3A to 3C and the workstation 3D for each computer 3, as described above.

2. Procedure

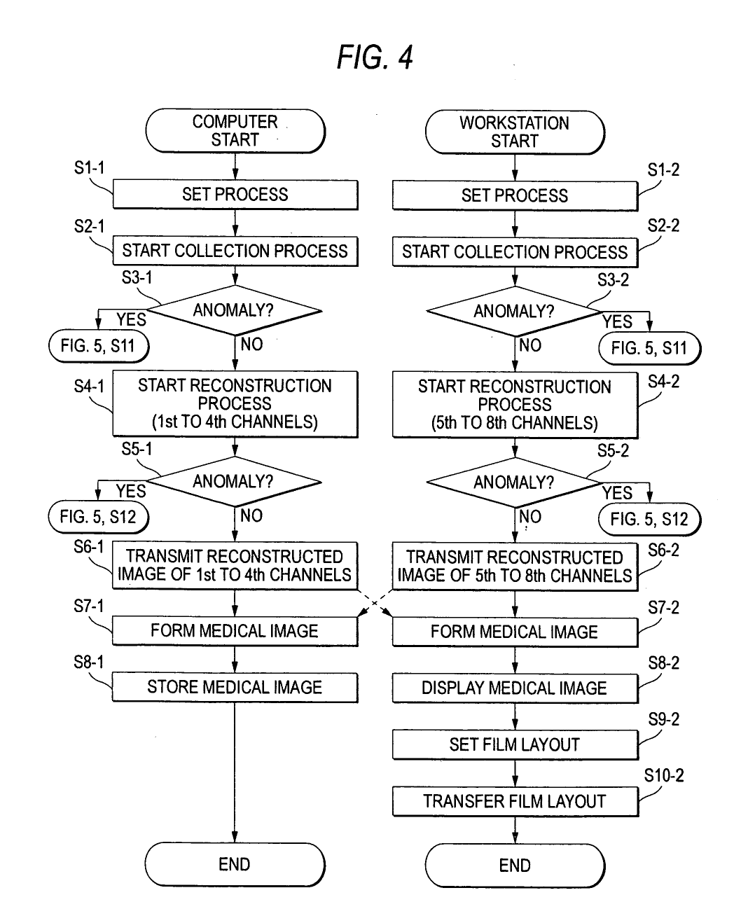

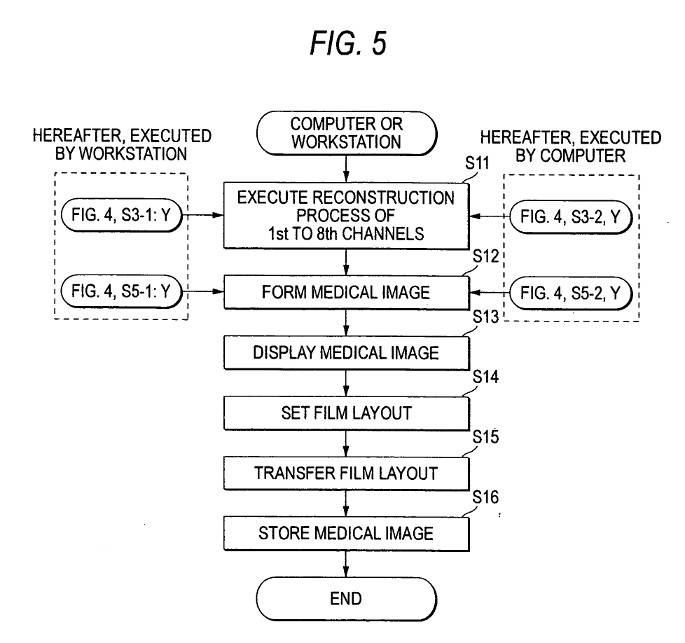

An example of the process executed by the diagnostic imaging system 1 will be described with reference to the flowchart of FIGS. 4 and 5.

In the following procedure, assume that the gantry 2 is an 8-channel MRI apparatus, that anomalies of the computers 3B and 3C have been detected in advance, and that the following process is executed using the two computers 3, the computer 3A and the workstation 3D.

In the flowchart of FIG. 4, the procedure by the computer 3A is indicated by Sn-1; the procedure by the workstation 3D is indicated by Sn-2. Although the flowchart of FIG. 4 shows only a data collecting process and a reconstructing process, the anomaly detection process by the anomaly detection section 41C is executed regularly as described above.

First, the processing-mode control section 41B of the management computer 4 sets a processing mode, sends a signal to the computer 3A and the workstation 3D, and then the computer 3A and the workstation 3D set the processes to be executed according to the set processing modes, respectively (S1-1, S1-2).

The processing-mode control section 41B sets the process, for example, in a default processing mode. When processing modes according to various processes and their set data are preset, the processing-mode control section 41B can select an appropriate mode from the preset processing modes. The processing modes can be preset using the user interface 36.

The details of the processing modes in Steps S1-1 and S1-2 are as follows: (1) the process of collecting data obtained by the gantry 2: a multiprocessing mode by the computer 3A and the workstation 3D; (2) a medial-image reconstructing process: a parallel processing mode for first to fourth channels (computer 3A) and fifth to eighth channels (workstation 3D); (3) a medical-image transfer process and a film layout process: a parallel processing mode by the computer 3A and the workstation 3D. When the two or more computers 3 actually execute different processes even in one process, as in the reconstructing process of (2), (the channels are different here), they correspond to “different processes”.

The set processing modes may be displayed on the display 34 of the workstation 3D to allow manual changes. Processing modes can be set manually or automatically according to exposure conditions, as described above.

The collecting sections 31B of the computer 3A and the workstation 3D start the process of collecting data that the gantry 2 obtained by scanning a subject, respectively (S2-1 S2-2). The timings of starting the collection are synchronized by the processing-mode control section 41B.

The anomaly detection section 41C of the management computer 4 regularly transmits an anomaly check signal to the computer 3A and the workstation 3D to detect whether an anomaly has occurred (S3-1, S3-2). The processing-state data returned in response to the anomaly check signal is recorded in the processing-state recording section 42B.

When an anomaly of either the computer 3A or the workstation 3D is detected during the collection process (S3-1, S3-2: Y), the processing-mode control section 41B sends a control signal to a computer 3 that has completed the collection process normally, allowing the subsequent processes to be executed singly (S11). The process by the single computer 3 will be described later.

On the other hand, when the collecting process has been completed without anomaly (S3-1, S3-2: N), the process moves to a medical-image reconstructing process (S4-1, S4-2). At that time, the timings of starting the reconstructing process are synchronized by the processing-mode control section 41B.

The computer 3A starts the reconstructing process for first to fourth channels of the eight receiving channels of the gantry 2 (MRI apparatus) (S4-1). The workstation 3D starts the reconstructing process for the fifth to eighth channels (S4-2).

The anomaly detection section 41C transmits an anomaly check signal to detect whether an anomaly has occurred in the computer 3A and the workstation 3D (S5-1, S5-2). The processing-state data that is returned in response to the anomaly check signal is recorded in the processing-state recording section 42B.

When an anomaly of either the computer 3A or the workstation 3D is detected during the reconstructing process (S5-1, S5-2: Y), the processing-mode control section 41B sends a control signal to a computer 3 that has completed the reconstructing process normally, allowing the reconstructing process executed by the computer 3 in which anomaly was detected and the subsequent processes to be executed singly (S12, to be described later).

On the other hand, when the reconstructing process has been completed without anomaly (S5-1, S5-2: N), the computer 3A transmits the reconstructed images of the first to fourth channels to the workstation 3D (S6-1). The workstation 3D also transmits the reconstructed images of the fifth to eighth channels to the computer 3A (S6-2). The dotted-line arrow in FIG. 4 indicates the transfer of the reconstructed image (image data).

The workstation 3D receives the reconstructed images of the first to fourth channels from the computer 3A, and combines them with the images of the fifth to eighth channels reconstructed by itself to form a medical image (S7-2). The display control section 31D displays the formed medical image on the display 34 (S8-2). The layout section 31E sets the film layout of the formed medical image according to the instruction by the operator through the input device 33 or automatically (S9-2). The set film layout is displayed on the display 34 by the display control section 31D. The layout section 31E transfers the set film layout to a film printer (not shown) via the communicating section 35 to form a film (S10-2). Thus the process by the workstation 3D is completed.

In parallel with steps S7-2 to S10-2, the computer 3A receives the reconstructed images of the fifth to eighth channels from the workstation 3D, and combines them with the images of the first to fourth channels reconstructed by itself to form a medical image (S7-1). The computer 3A then transfers the medial image to an archive database (not shown) to store it (S8-1). Thus the process by the computer 3A is completed.

The case where an anomaly of the processing state of the computer 3A or the workstation 3D was detected will be described with reference to FIG. 5.

When a positive determination is made in step S3-1, i.e., an anomaly of the computer 3A is detected during the collection process, the processing-mode control section 41B transmits a control signal to the workstation 3D. The workstation 3D executes the reconstructing process for all the first to eighth channels of the gantry 2 in response to the control signal (S11); combines them to form a medical image (S12); displays the medical image on the display 34 (S13); sets a film layout (S14); transfers the film layout to the film printer (S15); and transfers the medical image to an archive database to store it (S16).

When an anomaly of the workstation 3D is detected during the collection process (S3-2: Y), the processing-mode control section 41B sends a control signal to the computer 3A. The computer 3A executes processes from the reconstructing process for all the first to eighth channels of the gantry 2 (S11) to the storage of the medial image (S16) in response to the control signal.

Likewise, when an anomaly of the computer 3A is detected during the reconstructing process (S5-1: Y), the processing-mode control section 41B sends a control signal to the workstation 3D. Upon reception of the control signal, the workstation 3D executes processes from the reconstructing process for all the first to eighth channels of the gantry 2 (S11) to the storage of the medial image (S16). On the other hand, an anomaly of the workstation 3D is detected during the reconstructing process (S5-2: Y), the processing-mode control section 41B transmits a control signal to the computer 3A. Upon reception of the control signal, the computer 3A executes processes from the reconstructing process for all the first to eighth channels of the gantry 2 (S11) to the storage of the medial image (S16).

When an anomaly of the computer 3A is detected during the procedure from S6-1 to S8-1 by the computer 3A, the processing-mode control section 41B transmits a control signal to the workstation 3D to allow the workstation 3D to execute the medical-image forming process, the medical-image display process, the film-layout setting process and transferring process, and the medical-image storage process singly. Likewise, when an anomaly of the workstation 3D is detected during the procedure from S6-2 to S10-2 by the workstation 3D, the processing-mode control section 41B transmits a control signal to the computer 3A to allow the computer 3A to execute the medical-image forming process, the medical-image display process, the film-layout setting process and transferring process, and the medical-image storage process singly.

Processing-state data returned in response to regularly sent anomaly check signals during the anomaly detection by the anomaly detection section 41C is recorded in the processing-state recording section 42B.

When an anomaly is detected in steps S3-1, S3-2, S5-1, and S5-2, the processing-mode control section 41B attaches error information to processing-state data returned at the detection of the anomaly, and the processing-state transmitting section 41D sends it to the server 100 together with the error information. Upon reception of the processing-state data to which the error information is attached, the server 100 stores it in a database (not shown), and displays an error message on the display of the remote-control terminal 200, or alternatively, may generate a warning beep.

In the foregoing procedure, the process of collecting data obtained by the gantry 2 and the medical-image reconstructing process based on the collected data are examples of medical-image forming process. The process of displaying a medical image on the display 34, the medical-image film-layout setting process, the process of transferring the film layout to the film printer, and the process of transferring a medical image to an archive database are examples of post processing to the formed medical image.

3. Advantages

With the diagnostic imaging system 1, a multiprocessing mode is set for processes for which certainty is required such as a collection process. Accordingly, even if one computer fails to interrupt the process, another computer can continue the process, thus increasing the certainty of the process. On the other hand, a parallel processing mode is set for processes for which high speed is required such as a reconstructing process, thus allowing high-speed processing.

“A plurality of different processes” set for the parallel processing mode include at least two types. A first type is a method in which the process is divided into multiple different processes, and which are executed in a parallel processing mode. The embodiment discloses an example of the first type, in which the reconstructing process for the first to eighth channels is divided into a reconstructing process for the first to fourth channels and a reconstructing process fore the fifth to eighth process, and the two reconstructing processes are processed simultaneously by the two computers 3A and 3D. A second type is a method of executing a plurality of independent processes in a parallel processing mode. The embodiment discloses an example of the second type, in which the medical-image transfer process and the film-layout process are simultaneously executed by the two computers 3A and 3D.

With the diagnostic imaging system 1, the processing-state data of the computers 3 is recorded in the management computer 4 every time the anomaly detection section 41C regularly detects anomaly. Thus, the processing state at the occurrence of anomaly can be recorded. Particularly, the processes of the computers 3 are synchronized in the multiprocessing mode. Thus, even if one computer fails, the processing-state data of at least the other computer 3 is recorded. Accordingly, with the diagnostic imaging system 1, a service engineer can grasp the processing state when the computer fails, allowing effective quick maintenance.

Furthermore, the computers 3A to 3D of the diagnostic imaging system 1 include those with different hardware structures and OSs. Accordingly, the occurrence of anomaly due to the same cause, such as a failure of the same hardware or infection to the same computer virus, can be prevented. This further improves the certainty and high-speed processing, and improves the reliability of the record on the processing state at the occurrence of anomaly.

The diagnostic imaging system 1 allows, in addition to the automatic processing-mode setting by the processing-mode control section 41B, manual processing-mode setting using the user interface 36. Thus, the operator can execute a desired process in a desired processing mode. Particularly, a default processing mode is not always suitable for all users, because the operating state of the diagnostic imaging system 1 is different from user to user. Accordingly, provision of a processing-mode setting unit such as the user interface 36 of the embodiment allows the achievement of processing state suitable for the operating site.

When an anomaly of the computer 3 is detected, processing-state data and error information are transmitted from the management computer 4 to the server 100, and a message indicative of the occurrence of the anomaly is displayed on the display of the remote-control terminal 200. Thus a maintenance-service provider is notified of the anomaly quickly. Since the processing-state data is also sent certainly, the processing state at the occurrence of anomaly can be grasped, allowing effective quick maintenance.

In such systems as the diagnostic imaging system 1, various settings including an option setting are generally made in each medical institution that operates them, so that in general, the anomaly generation state also varies from institution to institution. When the system fails, the software for controlling the system is corrected to return the system. However, the correction may cause new problems because of the option setting. In such a case, since the corrected software is generally used as it is, the new problems cause a new anomaly to bring the system down again.

According to the embodiment, even if the software of a computer is corrected, the operation of the software of the corrected computer can be checked using pseudo data or the like while executing the original function using an uncorrected computer. Thus the correction of the software does not hinder the original process. The corrected computer can also be used for the backup of the other computers (refer to modification 3, to be described later) until the software is determined to be normal.

The procedure of the embodiment has been described for the process using two computers 3. However, it is also possible to execute the multiprocessing and the parallel processing using three or more computers 3 in proportion to the number of the computers 3 of the system. For example, for the parallel processing mode, high-speed processing can be achieved in such a manner that the computer 3A executes the reconstructing process for the first and second channels; the computer 3B executes the reconstructing process for the third and fourth channels; the computer 3C executes the reconstructing process for the fifth and sixth channels; and the workstation 3D executes the reconstructing process for the seventh and eighth channels. For the multiprocessing mode, the data collecting process is executed by three or more computers to further improve the certainty of the process.

A combination of the multiprocessing mode and the parallel processing mode is possible. For example, high-certainty and high-speed processing can be achieved in such a way that the computers 3A and 3B execute the reconstructing process for the first to fourth channels and the computer 3C and the workstation 3D execute the reconstructing process for the fifth to eighth channels.

4. Modifications

The above-described structures are only examples for implementing the invention. Accordingly, modifications may be made within its spirit and scope of the invention.

4-1. Modification 1

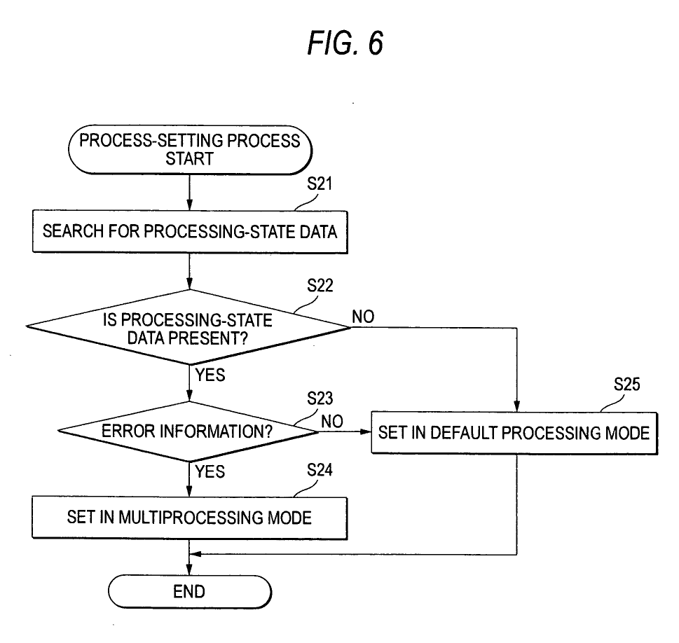

As a first modification, a structure will be described in which the certainty of the process can be improved with reference to processing-state data recorded in the processing-state recording section 42B. The first modification can be achieved by the procedure shown in FIG. 6 in the process setting process for a new process (Steps S1-1 and S1-2 in FIG. 4).

First, the processing-mode control section 41B searches the processing-state recording section 42B for processing-state data corresponding to a new process to be executed (e.g., a reconstructing process) (S21). Here the processing-state data corresponding to a new process indicates the same process as the new process (the reconstructing process). It is also possible to take into consideration not only to the sameness of the process but also to the sameness of the set data in the process (e.g., the kind of a reconstruction function being used).

When processing-state data corresponding to a new process is not found (S22: N), the processing-mode control section 41B sets a default processing mode (the default of the reconstructing process in FIG. 4 is a parallel processing mode) (S25), and terminates the process-setting process. When the operator changes the operating mode, the process is set to the processing mode.

On the other hand, when processing-state data corresponding to a new process is found (S22: Y), the processing-mode control section 41B determines whether the found processing-state data is accompanied by error information (S23). When no error information is attached (S23: N), the process is set to the default processing mode (S25), and the process-setting process is terminated. In step S25, the process may be set to a preset processing mode.

When error information is attached to the found processing-state data (S23: Y), the processing-mode control section 41B sets the processing mode of the new process to a multiprocessing mode (S24), and terminates the process-setting process.

Multiple pieces of processing-state data corresponding to the new process are sometimes found. In that case, the process is set to a multiprocessing mode when any of the multiple processing-state data is accompanied by error information. When it is determined, with reference to the time data attached to the processing-state data, that processing-state data in the last process has no error information and processing-state data in the preceding process has error information, the process can be set to the default processing mode. This is because the error so far is thought to be eliminated before the last process.

According to the modification 1, a process corresponding to processing-state data accompanied by error information is newly executed, in other word, a new process corresponding to the process in which anomaly was detected in the past is executed, the new process is controlled to be executed in a multiprocessing mode. This improves the certainty of the process in newly executing a process in which anomaly occurred in the past.

The accuracy for automatic setting to a multiprocessing mode can be improved by accumulating processing-state data when the computer 3 fails and by statistically analyzing the failed process and set data at that time. For example, the accumulated processing-state data is analyzed to obtain data on the frequency and number of the occurrence of anomalies, the frequency and number of the occurrence of anomalies at each step of process, and the processes before and after the occurrence of anomalies. For processes that fail with high frequency, a multiprocessing mode is always set, while for processes that fail with a frequency lower than a specified value, the processing mode is set manually using the data. This control can improve the accuracy of determination whether to automatically set in the multiprocessing mode.

4-2. Modification 2

In modification 1, when a process corresponding to a process that has failed during execution is executed, the processing mode for the new process is automatically set in a multiprocessing mode. The setting of the processing mode may be executed by an operator.

For example, when found processing-state data is accompanied by error information in step S23 of the flowchart of FIG. 6 (S23: Y), a message for setting the processing mode for the new process in a multiprocessing mode can be displayed on the display 34 (display unit) by the display control section 31D of the workstation 3D or the like that an operator is using. The message is, e.g., “the process may cause an error. Set it in a multiprocessing mode”. A warning beep may be output together with the message to encourage the operator to set the processing mode.

The operator who saw the message operates the input device 33 to set the processing mode in a multiprocessing mode. When anomalies are eliminated by maintenance before the new process, there is no need to set in the multiprocessing mode, but may be set in a parallel processing mode for high speed processing.

4-3. Modification 3

In the embodiment, when an anomaly of the computer 3 is detected, processing-state data and error information are stored in the processing-state recording section 42B of the management computer 4 and the database of the server 100. Modification 3 provides variations of storage of processing-state data and so on.

As shown in FIG. 2, the computers 3A to 3C and the workstation 3D each have the storage section 32. For example, if an anomaly of the computer 3A is detected with the computer 3A and the workstation 3D in operation, the management computer 4 attaches error information to processing-state data returned at the detection of the anomaly and sends it to the workstation 3D. The workstation 3D stores the received processing-state data and error information in the storage section 32.

In modification 3, as described above, the processing-state data on a failed computer is stored in a computer having no anomaly. Thus, even if an operator and a service engineer cannot access the management computer 4 because of an anomaly of a local network, they can grasp the processing state under an abnormal condition by referring to the processing-state data stored in the computer in a normal condition.

A backup computer may be provided to back up processing-state data, error information, and the results of processing by the computers. It is preferable that the backup computer have an OS or a program with high resistance to computer virus.

Alternatively, the processing-state data of one computer may be stored in the other computer when two or more computers are in operation. For example, when the computer 3A and the workstation 3D are in operation, the processing-state data and the process results of the computer 3A are transmitted to the workstation 3D to store them in its storage section 32, and the processing-state data and the process results of the workstation 3D are transmitted to the computer 3A to store them in its storage section 32. Such a mutual storing process may be performed regularly. Thus, even if any of two or more computers fails, the processing-state data and the process results of the failed computer are stored in a computer without failure. This allows maintenance with reference to the processing-state data, and the stored processing-state data to be used for the following processes. It is preferable to store the processing-state data of the computers also in the storage section 32 of the computer itself.

Processing by three or more computers 3 is the same as that by the two computers. For example, for the processing by three computers, processing-state data etc. by a first computer can be stored in the storage section 32 of a second computer, processing-state data etc. by the second computer can be stored in the storage section 32 of a third computer, and processing-state data etc. by the third computer can be stored in the storage section 32 of the first computer. The processing-state data etc. of the first computer may be stored in the second and third computers. The storage state of the processing-state data etc. for the case where the process is executed by three or more computers 3 may be set freely.

In contrast, one computer 3 may be used virtually as a plurality of computers. Specifically, one computer 3 can have the function of a plurality of computers by setting multiple areas in the storage section 32 of one computer 3 and operating the CPU 31 in parallel for the areas. Also in this case, the foregoing process can be executed using at least two or more virtual computers of one or more computer 3.

Accordingly, the “plurality of computers” includes a plurality of virtual computers, i.e., a plurality of virtual computers that are not physically separated from one another.

What is claimed is:

1. A diagnostic imaging system comprising:
a scanner that scans a subject to obtain data;
a plurality of computers; and
a control unit that switches between a multiprocessing mode and a parallel processing mode, the multiprocessing mode allowing the same process in any of a medical-image forming process based on the data obtained and the subsequent processes to be executed by two or more computers of the plurality of computers, and the parallel processing mode allowing a plurality of different processes in the medical-image forming process and the subsequent processes to be executed concurrently by two or more computers of the plurality of computers.

2. The diagnostic imaging system according to claim 1, further comprising:
a processing-mode setting unit that sets whether the process in any of the medical-image forming process and the subsequent processes is executed in the multiprocessing mode or the parallel processing mode, wherein
the control unit allows the two or more computers to execute the process in the set processing mode.

3. The diagnostic imaging system according to claim 1, further comprising:
an exposure-condition acquisition unit that acquires the exposure conditions of the scan, wherein
the control unit switches between the multiprocessing mode and the parallel processing mode depending on the exposure conditions acquired by the exposure-condition acquisition unit.

4. The diagnostic imaging system according to claim 1, further comprising:
an anomaly-detection unit that detects whether an anomaly has occurred in the processing state of the process in any of the medical-image forming process and the subsequent processes by the two or more computers; and
a processing-state recording unit that records the processing state when the anomaly is detected together with anomaly-detection information, wherein
when a new process for the processing state recorded together with the anomaly-detection information is executed, the control unit allows the two or more computers to execute the new process in the multiprocessing mode.

5. The diagnostic imaging system according to claim 1, wherein
a part of the plurality of computers has an operating system different from that of the other computers.

6. The diagnostic imaging system according to claim 1, wherein
a part of the plurality of computers has a hardware structure different from that of the other computers.

7. The diagnostic imaging system according to claim 2, further comprising:
an anomaly-detection unit that detects whether an anomaly has occurred in the process in any of the medical-image forming process and the subsequent processes by the two or more computers;
a processing-state recording unit that records the processing state when the anomaly is detected together with anomaly-detection information; and
a display unit that displays a message for setting the processing mode of a new process to the multiprocessing mode when the new process is executed, the new process corresponding to the processing state recorded together with the anomaly-detection information.

8. The diagnostic imaging system according to claim 3, wherein:
the exposure-condition acquisition unit acquires exposure conditions including whether a contrast medium is used; and
the control unit switches between the multiprocessing mode and the parallel processing mode depending on whether the contrast medium is used.

9. The diagnostic imaging system according to claim 4, wherein:
the anomaly-detection unit regularly detects whether the processing state of the process has failed, wherein
every time the anomaly-detection unit executes the regular detection, the processing state of the two or more computers is recorded in the processing-state recording unit.

10. The diagnostic imaging system according to claim 4, further comprising
a processing-state transmission unit connected to a remote computer that provides maintenance service for the plurality of computers via a network,
the processing-state transmission unit transmitting the processing state when the anomaly is detected by the anomaly-detection unit and the anomaly-detection information to the remote computer via the network.

11. The diagnostic imaging system according to claim 4, wherein
the processing-state recording unit is provided in each of the plurality of computers, wherein
the processing state when the anomaly is detected in any of the plurality of computers by the anomaly-detection unit and the anomaly-detection information are recorded in the processing-state recording unit disposed in the computer other than the failed computer.

12. A magnetic resonance imaging apparatus comprising:
a scanner that scans a subject to obtain data;
a plurality of computers;
an exposure-condition acquisition unit that acquires exposure conditions of the scan; and
a control unit that switches between a multiprocessing mode and a parallel processing mode depending on the exposure conditions, the multiprocessing mode allowing the same process in any of a medical-image forming process based on the data obtained and the subsequent processes to be executed by two or more computers of the plurality of computers, and the parallel processing mode allowing a plurality of different processes of any of the medical-image forming process and the subsequent processes to be executed concurrently by two or more computers of the plurality of computers.

13. The magnetic resonance imaging apparatus according to claim 12, wherein
the exposure-condition acquisition unit acquires exposure conditions including whether the scan is a dynamic scan, wherein
the control unit switches between the multiprocessing mode and the parallel processing mode depending on whether the scan is the dynamic scan.

14. The magnetic resonance imaging apparatus according to claim 12, wherein
the exposure-condition acquisition unit acquires exposure conditions including whether the scan is a whole-body scan, wherein
the control unit switches between the multiprocessing mode and the parallel processing mode depending on whether the scan is the whole-body scan.

15. The magnetic resonance imaging apparatus according to claim 12, wherein
the exposure-condition acquisition unit acquires exposure conditions including whether the scan is a moving-bed scan, wherein
the control unit switches between the multiprocessing mode and the parallel processing mode depending on whether the scan is the moving-bed scan.

16. The magnetic resonance imaging apparatus according to claim 12, wherein
the exposure-condition acquisition unit acquires exposure conditions including a pulse sequence, wherein
the control unit switches between the multiprocessing mode and the parallel processing mode depending on the pulse sequence.

17. The magnetic resonance imaging apparatus according to claim 16, wherein
the exposure-condition acquisition unit switches between the multiprocessing mode and the parallel processing mode depending on whether the pulse sequence is a diffusion sequence.

18. The magnetic resonance imaging apparatus according to claim 16, wherein
the exposure-condition acquisition unit switches between the multiprocessing mode and the parallel processing mode depending on whether the pulse sequence is a sequence belonging to angio imaging.

19. A method of diagnostic imaging, comprising the steps of:
obtaining data by scanning a subject; and
switching between a multiprocessing mode and a parallel processing mode, the multiprocessing mode allowing the same process in any of a medical-image forming process based on the data obtained and the subsequent processes to be executed by two or more computers of the plurality of computers, and the parallel processing mode allowing a plurality of different processes in the medical-image forming process and the subsequent processes to be executed concurrently by two or more computers of the plurality of computers.

20. The method of diagnostic imaging according to claim 19, further comprising the steps of:
setting whether the process in any of the medical-image forming process and the subsequent processes is executed in the multiprocessing mode or the parallel processing mode, wherein
the two or more computers are allowed to execute the process in the set processing mode.

21. The method of diagnostic imaging according to claim 19, further comprising the steps of:
acquiring the exposure conditions of the scan, wherein
the multiprocessing mode and the parallel processing mode are switched depending on the exposure conditions acquired.

22. The method of diagnostic imaging according to claim 19, further comprising the steps of:
detecting whether an anomaly has occurred in the processing state of the process in any of the medical-image forming process and the subsequent processes by the two or more computers; and
recording the processing state when the anomaly is detected together with anomaly-detection information, wherein
when a new process for the processing state recorded together with the anomaly-detection information is executed, the two or more computers are allowed to execute the new process in the multiprocessing mode.

23. The method of diagnostic imaging according to claim 19, wherein
a part of the plurality of computers has an operating system different from that of the other computers.

24. The method of diagnostic imaging according to claim 19, wherein
a part of the plurality of computers has a hardware structure different from that of the other computers.

25. The method of diagnostic imaging according to claim 20, further comprising the steps of:
detecting whether an anomaly has occurred in the process in any of the medical-image forming process and the subsequent processes by the two or more computers;
recording the processing state when the anomaly is detected together with anomaly-detection information; and
displaying a message for setting the processing mode of a new process to the multiprocessing mode when the new process is executed, the new process corresponding to the processing state recorded together with the anomaly-detection information.

26. The method of diagnostic imaging according to claim 21, wherein
exposure conditions including whether a contrast medium is used is acquired; and
the multiprocessing mode and the parallel processing mode are switched depending on whether the contrast medium is used.

27. The method of diagnostic imaging according to claim 22, wherein
it is regularly determined whether the processing state of the process has failed, wherein
every time the anomaly-detection unit executes the regular detection, the processing state of the two or more computers is recorded in the processing-state recording unit.

28. The method of diagnostic imaging according to claim 22, further comprising the steps of:
transmitting the processing state when the anomaly is detected and the anomaly-detection information to a remote computer via a network, the remote computer providing maintenance service for the plurality of computers via the network.

29. The method of diagnostic imaging according to claim 22, wherein
the processing state when the anomaly is detected in any of the plurality of computers by the anomaly-detection unit and the anomaly-detection information are recorded in the computer other than the failed computer.
